(19) United States
(12) Patent Application Publication
(10) Pub. No.: US 2005/0287092 A1
(43) Pub. Date: Dec. 29, 2005

(54) Cosmetic powder comprising barium sulfate

(76) Inventors: Anne Liechty, Paris (FR); Camille Thibout, Vincennes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/127,261

(22) Filed: May 12, 2005

Related U.S. Application Data
(60) Provisional application No. 60/617,676, filed on Oct. 13, 2004.

(30) Foreign Application Priority Data
May 13, 2004 (FR) 04 50933

(51) Int. Cl. A61K 7/021; A61K 35/00

(52) U.S. Cl. 424/63

(57) ABSTRACT

Disclosed herein is a cosmetic powder comprising barium sulfate and either at least one elastomeric organopolysiloxane powder and at least one N-acylamino acid powder, or at least one polymethyl methacrylate powder and at least one polyurethane powder, or at least one acrylic polymer powder other than a polymethyl methacrylate powder. Processes for the use of such powder for making up and/or caring for the skin are also disclosed.

This application claims benefit of U.S. Provisional Application No. 60/617,676, filed Oct. 13, 2004, the contents of which are incorporated herein by reference. This application also claims benefit of priority under 35 U.S.C. § 119 to French Patent Application No. 04 50933, filed May 13, 2004, the contents of which are also incorporated by reference.

Disclosed herein is a cosmetic makeup and/or skincare composition in powder form comprising barium sulfate combined with at least one other powder. Further disclosed herein is a process for making up and/or caring for human skin comprising applying the composition to the skin.

The makeup composition disclosed herein may be a skin makeup composition, such as a foundation, an eyeshadow, a makeup rouge, a concealer product, a face and body powder, and/or a body makeup product. For example, the composition may be a foundation composition.

The skincare composition may be a facial care product, a body care product, and/or a deodorant powder product.

Skin makeup compositions are commonly used to give an attractive color to the skin, such as the face, but also to mask skin imperfections, such as redness, marks, and wrinkles.

Certain makeup compositions are in the form of loose powder or compacted powder. These compositions may comprise a high content of powders, for example at least 80% by weight, relative to the total weight of the composition. Depending on the type of powders used, the cosmetic properties of the makeup product may be variable. However, certain powders, for instance calcium carbonate, magnesium carbonate, magnesium stearate, and titanium dioxide, give a dry, coarse feel and may be detrimental in terms of obtaining softness properties when the cosmetic powder is taken up on the finger or spread onto the skin. Furthermore, with these pulverulent materials, the deposit of the cosmetic powder on the skin may be opaque and covering, and therefore may not make it possible to obtain a transparent and masking makeup result and/or a makeup result that fades out the imperfections of the skin relief, for instance microreliefs, wrinkles, and fine lines. The powder deposited on the skin may be visible and may have a pronounced powdery appearance unlike the natural grain of the skin. Thus, the makeup result may not look natural.

Thus, it would be desirable to provide a makeup and/or skincare composition in powder form that has good properties in terms of softness when applied to the skin and makes it possible to obtain on the skin a deposit, such as a makeup deposit on natural skin, which is relatively non-powdery and which allows the natural grain of the skin to show through.

The present inventors have discovered that a composition having at least one of these properties may be obtained by combining a barium sulfate powder with other particular powders.

One embodiment disclosed herein is, for example, a cosmetic composition in powder form comprising barium sulfate, at least one elastomeric organopolysiloxane powder, and at least one N-acylamino acid powder.

Another embodiment disclosed herein is a cosmetic composition in powder form comprising barium sulfate, at least one polymethyl methacrylate powder, and at least one polyurethane powder.

Yet another embodiment disclosed herein is a cosmetic composition in powder form comprising barium sulfate and at least one acrylic polymer powder other than a polymethyl methacrylate powder.

Another embodiment disclosed herein is a non-therapeutic cosmetic process for making up and/or treating the skin, comprising applying to the skin a composition as disclosed herein.

Another embodiment disclosed herein is the use of a composition as defined above to obtain a deposit, such as a makeup deposit, on natural skin which may allow the grain of the skin to show through.

The composition disclosed herein comprises barium sulfate. The barium sulfate particles may be coated with an N-acylamino acid such as those described above. For example, the barium sulfate particles may be coated with lauroyllysine.

The barium sulfate may be present in the composition in an amount ranging from 1% to 10% by weight, such as ranging from 2% to 8% by weight or ranging from 3% to 7% by weight, relative to the total weight of the composition.

The composition may comprise at least one elastomeric organopolysiloxane powder, which may be spherical. Such a powder may make it possible to obtain a deposit, such as a makeup deposit, which is transparent, which masks the relief defects of the skin, and which is natural, leaving the natural grain of the skin to show through.

The at least one elastomeric organopolysiloxane powder may be crosslinked and may be obtained via a crosslinking addition reaction of diorganopolysiloxane containing at least one hydrogen linked to silicon and of diorganopolysiloxane containing ethylenically unsaturated groups linked to silicon; in at least one embodiment, this occurs in the presence of a platinum catalyst. Or the elastomeric organopolysiloxane may be obtained via a dehydrogenation crosslinking condensation reaction between a diorganopolysiloxane containing hydroxyl end groups and a diorganopolysiloxane containing at least one hydrogen linked to silicon, which may be in the presence of an organotin; or via a crosslinking condensation reaction of a diorganopolysiloxane containing hydroxyl end groups and of a hydrolysable organopolysilane; or via thermal crosslinking of organopolysiloxane, which may be in the presence of an organoperoxide catalyst; or via crosslinking of organopolysiloxane by high-energy radiation such as gamma rays, ultraviolet rays, or an electron beam.

In certain embodiments, the at least one elastomeric organopolysiloxane powder is crosslinked and is obtained via a crosslinking addition reaction (A2) of diorganopolysiloxane containing at least two hydrogens each linked to a silicon, and (B2) of diorganopolysiloxane containing at least two ethylenically unsaturated groups linked to silicon, which may be in the presence (C2) of a platinum catalyst, for instance as described in European Patent Application No. EP A 295 886.

In certain embodiments, the organopolysiloxane may be obtained via a reaction of dimethylpolysiloxane containing dimethylvinylsiloxy end groups and of methylhydrogenopolysiloxane containing trimethylsiloxy end groups, in the presence of a platinum catalyst.

Compound (A2) is the base reagent for the formation of elastomeric organopolysiloxane and the crosslinking takes place via an addition reaction of compound (A2) with compound (B2) in the presence of the catalyst (C2).

Compound (A2) may be a diorganopolysiloxane containing at least two lower alkenyl groups (for example C₂-C₄). The lower alkenyl group may be chosen from vinyl, allyl, and propenyl groups. These lower alkenyl groups may be located in any position on the organopolysiloxane molecule, for example, they may be located at the ends of the organopolysiloxane molecule. The organopolysiloxane (A2) may have a branched-chain, linear-chain, cyclic, or network structure. For example, the organopolysiloxane (A2) may have a linear-chain structure. Compound (A2) may have a viscosity ranging from the liquid state to the gum state. For example, compound (A2) may have a viscosity of at least 100 centistokes at 25° C.

The organopolysiloxanes (A2) may be chosen from methylvinylsiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethyl-siloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxane containing dimethylvinylsiloxy end groups, and dimethylsiloxane-methyl(3,3,3-trifluoropropyl)siloxane copolymers containing dimethylvinylsiloxy end groups.

Compound (B2) may be an organopolysiloxane containing at least two hydrogens linked to silicon in each molecule, and thus may be the crosslinking agent for the compound (A2).

In certain embodiments, the sum of the number of ethylenic groups per molecule of compound (A2) and the number of hydrogen atoms linked to silicon per molecule of compound (B2) is at least 4.

Compound (B2) may be of any molecular structure, for example of linear-chain, branched-chain, or cyclic structure.

Compound (B2) may have a viscosity at 25° C. ranging from 1 to 50,000 centistokes, which may aid in having good miscibility with compound (A).

In certain embodiments, compound (B2) may be added in an amount such that the molecular ratio between the total amount of hydrogen atoms linked to silicon in compound (B2) and the total amount of all the ethylenically unsaturated groups in compound (A2) ranges from 1/1 to 20/1.

Compound (B2) may be chosen from methylhydrogenopolysiloxanes containing trimethylsiloxy end groups, dimethylsiloxane-methylhydrogenosiloxane copolymers containing trimethylsiloxy end groups, and cyclic dimethylsiloxane-methylhydrogenosiloxane copolymers.

Compound (C2) is the crosslinking reaction catalyst, and may be chosen from chloroplatinic acid, chloroplatinic acid-olefin complexes, chloroplatinic acid-alkenylsiloxane complexes, chloroplatinic acid-diketone complexes, platinum black, and platinum on a support.

The catalyst (C2) may be added in an amount ranging from 0.1 to 1,000 parts by weight, such as from 1 to 100 parts by weight, as clean platinum metal, per 1,000 parts by weight of the total amount of compounds (A2) and (B2).

Other organic groups may be linked to silicon in the organopolysiloxanes (A2) and (B2) described above, for instance alkyl groups such as methyl, ethyl, propyl, butyl, and octyl; substituted alkyl groups such as 2-phenylethyl, 2-phenylpropyl, and 3,3,3-tri-fluoropropyl; aryl groups such as phenyl, tolyl, and xylyl; substituted aryl groups such as phenylethyl; and substituted monovalent hydrocarbon-based groups such as epoxy groups, carboxylate ester groups, and mercapto groups.

The elastomeric organopolysiloxane may be non-emulsifying. As used herein, the term “non-emulsifying” denotes organopolysiloxane elastomers not containing a hydrophilic chain such as polyoxyalkylene or polyglycerolated units.

Spherical elastomeric organopolysiloxanes are described, for example, in Japanese Patent Application No. JP A 61 194 009 and European Patent Application Nos. EP A 242 219, EP A 295 886, and EP A 765 656, the contents of which are incorporated herein by reference.

The at least one elastomer organopolysiloxane powder that may be used include those sold under the names Dow Corning 9505 Powder and Dow Corning 9506 Powder by the company Dow Coming. These powders have the INCI name dimethicone/vinyl dimethicone crosspolymer.

The at least one elastomeric organopolysiloxane powder may comprise at least one elastomeric organopolysiloxane powder coated with silicone resin, for example coated with silsesquioxane resin, as described, for example, in U.S. Pat. No. 5,538,793, the content of which is incorporated herein by reference. Such elastomeric powders are sold under the names KSP-100, KSP-101, KSP-102, KSP-103, KSP-104, and KSP-105 by the company Shin-Etsu and have the INCI name vinyl dimethicone/methicone silsesquioxane crosspolymer.

Other elastomeric organopolysiloxanes in the form of spherical powders may be powders of hybrid silicone functionalized with fluoroalkyl groups, sold for example under the name KSP-200 by the company Shin-Etsu and powders of hybrid silicones functionalized with phenyl groups, sold for example under the name KSP-300 by the company Shin-Etsu.

Thus, the composition may comprise at least two powders of elastomeric organopolysiloxane containing at least one elastomeric organopolysiloxane powder coated with silicone resin, for example coated with silsesquioxane resin, as described above.

The at least one elastomeric organopolysiloxane powder, such as a non-emulsifying elastomeric organopolysiloxane, which may be spherical, may be present in the composition in an amount ranging from 5% to 25% by weight, such as from 7% to 15% by weight, or from 8% to 12% by weight, relative to the total weight of the composition.

In certain embodiments, the composition disclosed herein may comprise an elastomeric organopolysiloxane powder coated with silicone resin, for example coated with silsesquioxane resin, in an amount ranging from 1% to 10% by weight, such as from 2% to 8% by weight or from 3% to 7% by weight, relative to the total weight of the composition.

The composition may comprise a mixture of elastomeric organopolysiloxane powder coated with silicone resin, for example coated with silsesquioxane resin, and of uncoated elastomeric organopolysiloxane powder. In such a mixture, the elastomeric organopolysiloxane powder coated with silicone resin, for example coated with silsesquioxane resin, may be present in an amount ranging from 1% to 10% by weight, such as from 2% to 8% by weight or from 3% to 7% by weight, relative to the total weight of the composition. The uncoated elastomeric organopolysiloxane powder may be present in an amount ranging from 1% to 10% by weight, such as from 2% to 8% by weight or from 3% to 7% by weight, relative to the total weight of the composition.

The composition disclosed herein may comprise at least one spherical filler. This at least one spherical filler may be chosen from mineral fillers and organic fillers. This at least one spherical filler is different from the at least one elastomeric organopolysiloxane powder described above. This at least one spherical filler may be non-elastomeric. In certain embodiments, this at least one spherical filler is not film-forming, i.e., it does not form a continuous film when it is deposited onto a support such as the skin.

The at least one spherical filler may be chosen, for example, from:
acrylic powders, such as polymethyl methacrylate powders;
acrylic powders other than polymethyl methacrylate powders; and
polyurethane powders,
   these powders being described below.

The composition disclosed herein may comprise at least one spherical filler in an amount ranging from 10% to 40% by weight, such as from 15% to 35% by weight, from 15% to 30% by weight, or from 15% to 25% by weight, relative to the total weight of the composition.

According to one embodiment disclosed herein, the composition may comprise a powder, such as a spherical powder, of polymethyl methacrylate.

The polymethyl methacrylate powders may be in the form of hollow or solid white spherical particles, which may have a number-average size of micrometer order, such as a size ranging from 3 to 15 microns or from 3 to 10 microns. As used herein, the expression “number-average size” denotes the size given by the statistical particle size distribution to half of the population, referred to as D50.

It is also possible to characterize these polymethyl methacrylate particles by their density, which can vary as a function of the size of the spherical cavity of the said particles.

In the context of certain embodiments, this density is assessed according to the following protocol, referred to as the packed density:

A quantity of powder (m), wherein m may be, for example, 40 g, is poured into a measuring cylinder, and the measuring cylinder is then placed on a Stav 2003 machine from Stampf Volumeter. The measuring cylinder is then subjected to 1,500 packing motions. The final volume Vf of packed powder is then measured directly on the measuring cylinder. The packed density is determined by the ratio m/Vf, in this instance 40/Vf (Vf being expressed in cm³ and m being expressed in g).

In certain embodiments, the density of the polymethyl methacrylate particles that may be used may range from 0.3 to 1.5, such as from 0.5 to 1.5 or from 1 to 1.5.

As non-limiting illustrations of the polymethyl methacrylates that are suitable for certain embodiments, mention may be made of the polymethyl methacrylate particles sold by the company Matsumoto Yushi Co. under the name Micropearl M100, particles sold by the company LCW under the name Covabead LH 85, and particles sold by the company Nihon Junyaku under the name Jurymer MB1.

The polymethyl methacrylate particles may be present in an amount ranging from 5% to 20% by weight, such as from 7% to 18% by weight or from 8% to 15% by weight, relative to the total weight of the composition.

The composition disclosed herein may comprise at least one powder, such as at least one spherical powder, of an acrylic polymer, other than a polymethyl methacrylate powder.

The acrylic powder may be an acrylonitrile polymer or copolymer powder, such as expanded hollow particles of acrylonitrile polymer or copolymer. The particles may be made of any expanded acrylonitrile polymer or copolymer that is non-toxic and non-irritating to the skin.

In certain embodiments, the mass per unit volume of the particles may range from 15 kg/m³ to 200 kg/m³, such as from 40 kg/m³ to 120 kg/m³ or from 60 kg/m³ to 80 kg/m³. To obtain this low mass per unit volume, expanded polymer or copolymer particles, such as particles based on acrylonitrile and on an acrylic or styrene monomer and/or on vinylidene chloride, may be used.

It is possible to use, for example, a copolymer containing from 0% to 60% of units derived from vinylidene chloride, from 20% to 90% of units derived from acrylonitrile, and from 0% to 50% of units derived from an acrylic or styrene monomer, the sum of the percentages (by weight) being equal to 100. The acrylic monomer may be chosen from methyl acrylate, ethyl acrylate, and methacrylate. The styrene monomer may be chosen from α-methylstyrene and styrene.

The particles used herein may be hollow particles of an expanded copolymer of vinylidene chloride and of acrylonitrile or an expanded polymer of vinylidene chloride, of acrylonitrile, and of methacrylate. These particles may be dry or hydrated.

The particles disclosed herein may be obtained, for example, according to the processes disclosed in the European Patent and Patent Application Nos. EP 56 219, EP 348 372, EP 486 080, EP 320 473, and EP 112 807, and U.S. Pat. No. 3,615,972.

The internal cavity of the particles may contain at least one gas, which may be chosen from air, nitrogen, and hydrocarbons, for instance isobutane or isopentane.

The particles disclosed herein may have a particle size ranging from 1 μm to 80 μm, such as from 10 μm to 50 μm or from 10 μm to 30 μm.

The particles that may be used according to certain embodiments are, for example, expanded terpolymer microspheres of vinylidene chloride, of acrylonitrile and of methacrylate, sold under the brand name Expancel® by the company Expancel under the references 551 DE 50 (particle size of about 40 μm), 551 DE 20 (particle size of about 30 μm and mass per unit volume of about 65 kg/m³), 551 DE 12 (particle size of about 12 μm), 551 DE 80 (particle size of about 80 μm), and 461 DE 50 (particle size of about 50 μm). It is also possible to use microspheres formed from the same expanded terpolymer having a particle size of about 18 μm and a mass per unit volume of about 70 kg/m³, referred to hereinafter as EL 23, or having a particle size of about 34 μm and a mass per unit volume of about 20 kg/m³, referred to hereinafter as EL 43.

In certain embodiments, the acrylic powder other than polymethyl methacrylate is present in the composition when it is in the form of a loose powder.

The acrylic powder other than polymethyl methacrylate powder may be present in the composition disclosed herein in an amount ranging from 0.05% to 2% by weight, such as from 0.1% to 1.5% by weight or from 0.1% to 1.2% by weight, relative to the total weight of the composition.

The composition disclosed herein may comprise a powder, such as at least one spherical powder, of polyurethane. In certain embodiments, the at least one polyurethane powder is not film-forming, i.e., it does not form a continuous film when it is deposited onto a support such as the skin.

The at least one polyurethane powder may be a powder of a copolymer of hexamethylene diisocyanate and of trimethylol hexyl lactone. Such a polyurethane powder is sold for example under the names Plastic Powder D-400 and Plastic Powder D-800 by the company Toshiki.

Other polyurethane powders that may be used include the product sold under the name Plastic Powder CS-400 by the company Toshiki.

The at least one polyurethane powder may be present in the composition disclosed herein in an amount ranging from 0.5% to 30% by weight, such as from 1% to 15% by weight or from 5% to 15% by weight, relative to the total weight of the composition.

The composition disclosed herein may comprise an N-acylamino acid powder. Such a powder may give the cosmetic powder a creamy property.

The N-acylamino acids may comprise an acyl group containing from 8 to 22 carbon atoms, for instance the acyl group may be chosen from 2-ethylhexanoyl, caproyl, lauroyl, myristoyl, palmitoyl, stearoyl, and cocoyl groups. The amino acid may be, for example, chosen from lysine, glutamic acid, and alanine.

The N-acylamino acid powder may, for example, be a lauroyllysine powder.

The N-acylamino acid powder may be present in the composition disclosed herein in an amount ranging from 5% to 20% by weight, such as from 7% to 18% by weight or from 8% to 15% by weight, relative to the total weight of the composition.

The composition may comprise at least one pulverulent dyestuff, which may be chosen from pigments and nacres.

As used herein, the term “pigments” should be understood as meaning white or colored, mineral or organic particles of any shape, which may be insoluble in the physiological medium, and which are intended to color the composition.

The term “nacres” should be understood as meaning iridescent particles of any shape, for example particles produced in the shell of certain molluscs or alternatively synthesized.

The pigments may be white or colored, and mineral and/or organic. Among the mineral pigments that may be mentioned are titanium dioxide, optionally surface-treated, zirconium oxide, cerium oxide, zinc oxide, iron oxide (black, yellow, and red), chromium oxide, manganese violet, ultramarine blue, chromium hydrate, ferric blue, and metal powders, for instance aluminium powder and copper powder.

Among the organic pigments that may be mentioned are carbon black, D & C pigments, and lakes based on at least one of cochineal carmine, barium, strontium, calcium, and aluminium.

The nacreous pigments may be chosen from white nacreous pigments such as mica coated with titanium or with bismuth oxychloride, colored nacreous pigments such as titanium mica coated with iron oxides, titanium mica coated with ferric blue or with chromium oxide, titanium mica coated with an organic pigment of the abovementioned type, and nacreous pigments based on bismuth oxychloride.

It is also possible to use goniochromatic pigments. These pigments may exhibit a relatively large color change according to the angle of observation.

The goniochromatic pigment may be chosen, for example, from pigments of multilayer interference structure and liquid-crystal pigments.

In the case of a multilayer structure, this structure may comprise, for example, at least two layers, each layer, independently of the other layer(s) or otherwise, being made, for example, from at least one material chosen from the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, and alloys and polymers thereof.

The goniochromatic agents with multilayer structures are, for example, those described in the following patent documents: U.S. Pat. No. 3,438,796; European Patent No. EP A 227 423; U.S. Pat. No. 5,135,812; European Patent Nos. EP A 170 439 and EP A 341 002; U.S. Pat. Nos. 4,930,866 and 5,641,719; European Patent Nos. EP A 472 371; EP A 395 410; EP A 753 545; EP A 768 343; EP A 571 836; EP A 708 154; and EP A 579 091; U.S. Pat. No. 5,411,586 and 5,364,467; PCT Patent Application No. WO A 97/39066; German Patent No. DE A 4 225 031; PCT Patent Application No. WO 95/17479 (BASF); and German Patent No. DE A 196 14 637. The goniochromatic agents may be in the form of flakes of metallized color.

The multilayer structures that may be used in accordance with certain embodiments are, for example, chosen from the following structures: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica-oxide/SiO₂/MoS₂; and Fe₂O₃/SiO₂/mica-oxide/SiO₂/Fe₂O₃. Different colors may be obtained depending on the thickness of the various layers. Thus, with the structure Fe₂O₃/SiO₂/Al/SiO₂/Fe₂O₃, the color changes from green-golden to red-grey for SiO₂ layers of from 320 to 350 nm; from red to golden for SiO₂ layers of from 380 to 400 nm; from violet to green for SiO₂ layers of from 410 to 420 nm; and from copper to red for SiO₂ layers of from 430 to 440 nm.

The multilayer structure may be mineral or organic. Different colors may be obtained depending on the thickness of each of the various layers.

The goniochromatic pigments of multilayer interference structure as disclosed herein may be chosen from at least one of those described in the following patent documents: U.S. Pat. No. 3,438,796; European Patent No. EP A 227 423; U.S. Pat. No. 5,135,812; European Patent Nos. EP A 170 439 and EP A 341 002; U.S. Pat. Nos. 4,930,866 and 5,641,719; European Patent Nos. EP A 472 371; EP A 395 410; EP A 753 545; EP A 768 343; EP A 571 836; EP A 708 154; and EP A 579 091; U.S. Pat. Nos. 5,411,586 and 5,364,467; PCT Patent Application No. WO A 97/39066; German Patent No. DE A 4 225 031; PCT Patent Application No. WO 95/17479 (BASF); and German Patent No. DE A 196 14 637. They may be in the form of flakes of metallized color.

The goniochromatic pigment of multilayer interference structure may be chosen from at least one of the following commercial goniochromatic pigments: Infinite Colors from the company Shiseido, Sicopearl Fantastico from BASF, Colorstream, Xirallic and Xirona from Merck, and Colorglitter from Flex.

As goniochromatic pigments of multilayer structure, mention may be made of those sold under the name Sicopearl.

Liquid-crystal pigments are, for example, described in European Patent Application No. EP A 1 046 692.

Liquid-crystal particles that may be used include those known under the CTFA name Polyacrylate-4 and sold under the names Helicone® HC Sapphire, Helicone® HC Scarabeus, Helicone® HC Jade, Helicone® HC Maple, Helicone® HC XL Sapphire, Helicone® HC XL Scarabeus, Helicone® HC XL Jade, and Helicone® HC XL Maple by the company Wacker.

The dyestuffs may be present in the composition disclosed herein in an amount ranging from 0.5% to 40% by weight, such as from 1% to 30% by weight or from 3% to 25% by weight, relative to the total weight of the composition.

The composition disclosed herein may comprise at least one additional filler, other than the elastomeric organopolysiloxane powders and the at least one spherical filler described above.

As used herein, the term “fillers” should be understood as meaning colorless or white, mineral or synthetic particles of any shape, which are insoluble in the medium of the composition irrespective of the temperature at which the composition is manufactured.

The at least one additional filler may be mineral or organic and of any shape. For example, the at least one additional filler may be platelet-shaped, spherical, or oblong, irrespective of the crystallographic form (for example lamellar, cubic, hexagonal, orthorhombic, etc.). Mention may be made of talc, mica, silica, kaolin, polyamide (Nylon®) powders, poly-β-alanine powders, polyethylene powders, tetrafluoroethylene polymer (Teflon®) powders, starch, boron nitride, silicone resin powders (for example Tospearls® from Toshiba), hydroxyapatite, sericite, glass beads, and ceramic beads.

The at least one additional filler may be present in the composition in an amount ranging from 0.5% to 75% by weight, such as from 1% to 60% by weight or from 5% to 60% by weight, relative to the total weight of the composition.

The composition disclosed herein may be free of compounds chosen from calcium carbonate, magnesium carbonate, magnesium hydrocarbonate, chalks, titanium dioxide, metal soaps derived from organic carboxylic acids containing from 8 to 22 carbon atoms (such as from 12 to 18 carbon atoms), for example zinc stearate, magnesium stearate, lithium stearate, zinc laurate, and magnesium myristate. Such compounds may have the drawback of harming the soft feel of the cosmetic powder.

As used herein, the term “free of” means that the composition contains less than 4% by weight of the said compounds from which it is free, such as less than 3% by weight, less than 2% by weight, less than 1% by weight, or even does not contain any (i.e., 0% by weight) of such compounds, relative to the total weight of the composition.

The composition disclosed herein may comprise a total content of pulverulent compounds ranging from 80% to 99% by weight, such as from 85% to 99% by weight, relative to the total weight of the composition.

The composition disclosed herein may comprise at least one fatty phase, which may comprise at least one oil. This type of fatty phase is also commonly referred to as a binder and may serve as a dispersing medium for the particulate phase.

The oil may be chosen from the oils conventionally used as binder in loose or compact powders. These oils may be chosen from:

mink oil, turtle oil, soybean oil, grapeseed oil, sesame seed oil, corn oil, rapeseed oil, sunflower oil, cottonseed oil, avocado oil, olive oil, castor oil, jojoba oil, and groundnut oil;

hydrocarbon oils such as liquid paraffin, squalane, and petroleum jelly;

fatty esters, such as isopropyl myristate, isopropyl palmitate, butyl stearate, isodecyl stearate, isocetyl stearate, hexyl laurate, isononyl isononanoate, 2-ethylhexyl palmitate, 2-hexyldecyl laurate, 2-octyldecyl palmitate, 2-octyldodecyl myristate, 2-octyidodecyl lactate, 2-diethylhexyl succinate, diisostearyl malate, glyceryl triisostearate, and diglyceryl triisostearate;

silicone oils such as polymethylsiloxanes, polymethylphenylsiloxanes, polysiloxanes modified with fatty acids, polysiloxanes modified with fatty alcohols, polysiloxanes modified with polyoxyalkylenes, fluoro silicones, and perfluoro oils;

higher fatty acids such as myristic acid, palmitic acid, stearic acid, behenic acid, oleic acid, linoleic acid, linolenic acid, and isostearic acid;

higher fatty alcohols such as cetanol, stearyl alcohol, and oleyl alcohol;

poly methylfluoroalkyl dimethylsiloxanes of formula (I): 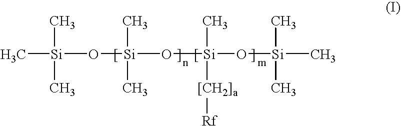
in which:

n is an integer ranging from 5 to 90, such as from 30 to 80 or from 50 to 80,

m is an integer ranging from 1 to 150, such as 1 to 80 or from 1 to 40,

a is an integer ranging from 0 to 5, and

Rf denotes a perfluoroalkyl radical containing from 1 to 8 carbon atoms;

and mixtures thereof.

Compounds of formula (I) that may be mentioned include those sold under the names X22-819, X22-820, X22-821, and X22-822 by the company Shin-Etsu.

The composition disclosed herein may comprise at least one oil in an amount ranging from 1% to 20% by weight, such as from 2% to 15% by weight, relative to the total weight of the composition.

The composition may comprise other common cosmetic ingredients, which may be chosen from antioxidants, fragrances, preserving agents, neutralizers, surfactants, waxes, water, sunscreens, vitamins, moisturizers, self-tanning compounds, and antiwrinkle active agents.

Needless to say, a person skilled in the art will take care to select this or these optional additional compound(s), and/or the amount thereof, such that the advantageous properties of the composition disclosed herein are not, or are not substantially, adversely affected by the envisaged addition.

The composition disclosed herein may be an anhydrous composition. As used herein, the term “anhydrous composition” means a composition containing less than 2% by weight of water, such as less than 0.5% of water or free of water, the water not being added during the preparation of the composition, but corresponding to the residual water provided by the mixed ingredients.

The composition disclosed herein may be in the form of a loose powder or a compact powder. As used herein, the term “compact powder” denotes a powder pressed using a manual or mechanical press.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result.

The invention is illustrated in greater detail by the examples described below.

### EXAMPLE 1

A loose face powder having the following composition was prepared:

| | | |
|---|---|---|
| Particles of crosslinked polydimethylsiloxane coated with | 5 | g |
| silicone resin (KSP-100 from the company Shin-Etsu) | | |
| Spherical powder of elastomeric silicone | 5 | g |
| (Dow Corning 9506 Powder from Dow Corning) | | |
| Polyurethane spherical powder | 10 | g |
| (Plastic Powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.5 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 37.6 | g |
| Mica | 15 | g |
| Iron oxides | 3.30 | g |
| Preserving agents | 0.6 | g |
| Isocetyl stearate | 3 | g |

The powder spread easily on the face and had good softness properties. The makeup result obtained was transparent, homogeneous, unifying, had a natural appearance that allowed the grain of the skin to show through, and faded out the relief defects of the skin.

### EXAMPLE 2

A loose face powder having the following composition was prepared:

| | | |
|---|---|---|
| Particles of crosslinked polydimethylsiloxane coated with | 5 | g |
| silicone resin (KSP-100 from the company Shin-Etsu) | | |
| Spherical powder of elastomeric silicone | 5 | g |
| (Dow Corning 9506 Powder from Dow Corning) | | |
| Polyurethane spherical powder | 10 | g |
| (Plastic Powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.5 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 12.7 | g |
| Mica | 12 | g |
| Iron oxides | 5.2 | g |
| Nacreous pigments | 26 | g |
| Preserving agents | 0.6 | g |
| Isocetyl stearate | 3 | g |

The powder spread easily on the face and had good softness properties. The makeup result obtained was transparent, homogeneous, unifying, had a natural appearance that allowed the grain of the skin to show through, and faded out the relief defects of the skin.

### EXAMPLE 3

A loose face powder having the following composition was prepared:

| | | |
|---|---|---|
| Particles of crosslinked polydimethylsiloxane | 20 | g |
| coated with silicone resin | | |
| (KSP-100 from the company Shin-Etsu) | | |
| Polyurethane spherical powder | 10 | g |
| (Plastic Powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.50 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 27.6 | g |
| Mica | 15 | g |
| Iron oxides | 3.30 | g |
| Preserving agents | 0.6 | g |
| Isocetyl stearate | 3 | g |

The powder spread easily on the face and had good softness properties. The makeup result obtained was transparent, homogeneous, unifying, had a natural appearance that allowed the grain of the skin to show through, and faded out the relief defects of the skin.

### EXAMPLE 4

A compact face powder having the following composition was prepared:

| | | |
|---|---|---|
| Particles of crosslinked polydimethylsiloxane coated with | 5 | g |
| silicone resin | | |
| (KSP-100 from the company Shin-Etsu) | | |
| Spherical elastomeric silicone powder (Dow Corning 9506 | 5 | g |
| powder from Dow Corning) | | |
| Polyurethane spherical powder | 10 | g |
| (plastic powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.50 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 21.2 | g |
| Mica | 10 | g |
| Iron oxides | 3.30 | g |
| Zinc stearate | 3 | g |
| Bismuth oxychloride | 10 | g |
| Preserving agents | 1.4 | g |
| Phenyl trimethicone | 6 | g |
| Glyceryl triisostearate | 6 | g |

The powder was screened, poured into a metal dish, and then compacted.

The powder spread easily on the face and had good softness properties. The makeup result obtained was transparent, homogeneous, unifying, had a natural appearance that allowed the grain of the skin to show through, and faded out the relief defects of the skin.

1. A cosmetic composition in powder form comprising
barium sulfate and
at least two powders chosen from one of the following combinations:
(A) at least one elastomeric organopolysiloxane powder and at least one N-acylamino acid powder;
(B) at least one polymethyl methacrylate powder and at least one polyurethane powder; and
(C) at least one acrylic polymer powder other than a polymethyl methacrylate powder.

2. A cosmetic composition in powder form according to claim 1, said composition comprising barium sulfate, at least one elastomeric organopolysiloxane powder and at least one N-acylamino acid powder.

3. A cosmetic composition in powder form according to claim 1, said composition comprising barium sulfate, at least one polymethyl methacrylate powder and at least one polyurethane powder.

4. A cosmetic composition in powder form according to claim 1, said composition comprising barium sulfate and at least one acrylic polymer powder other than a polymethyl methacrylate powder.

5. A cosmetic composition in powder form according to claim 1, wherein when the at least two powders are chosen from combination (B) or (C), the composition further comprises at least one elastomeric organopolysiloxane powder.

6. A composition according to claim 1, wherein the at least one elastomeric organopolysiloxane is obtained via at least one method chosen from:
a crosslinking addition reaction of diorganosiloxane containing at least one hydrogen linked to silicon and of diorganopolysiloxane containing ethylenically unsaturated groups linked to silicon;
a dehydrogenation crosslinking condensation reaction between a diorganopolysiloxane containing hydroxyl end groups and a diorganopolysiloxane containing at least one hydrogen linked to silicon;
a crosslinking condensation reaction of a diorganopolysiloxane containing hydroxyl end groups and of a hydrolysable organopolysilane;
thermal crosslinking of organopolysiloxane; and
crosslinking of organopolysiloxane by high-energy radiation.

7. The composition according to claim 1, wherein the at least one elastomeric organopolysiloxane is obtained via a crosslinking addition reaction (1) of diorganopolysiloxane containing at least two hydrogens each linked to a silicon, and (2) of diorganopolysiloxane containing at least two ethylenically unsaturated groups linked to silicon.

8. The composition according to claim 1, wherein the at least one elastomeric organopolysiloxane is obtained by reaction of dimethylpolysiloxane containing dimethylvinylsiloxy end groups and of methylhydrogenopolysiloxane containing trimethylsiloxy end groups, in the presence of a platinum catalyst.

9. The composition according to claim 1, wherein the at least one elastomeric organopolysiloxane is a non-emulsifying elastomer.

10. The composition according to claim 1, wherein the at least one elastomeric organopolysiloxane powder comprises at least one elastomeric organopolysiloxane powder coated with silicone resin.

11. The composition according to claim 1, wherein the at least one elastomeric organopolysiloxane powder is a spherical powder.

12. The composition according to claim 1, wherein the at least one elastomeric organopolysiloxane powder is present in an amount ranging from 5% to 25% by weight, relative to the total weight of the composition.

13. The composition according to claim 12, wherein the at least one elastomeric organopolysiloxane powder is present in an amount ranging from 8% to 12% by weight, relative to the total weight of the composition.

14. The composition according to claim 10, wherein the at least one elastomeric organopolysiloxane powder coated with silicone resin is present in an amount ranging from 1% to 25% by weight, relative to the total weight of the composition.

15. The composition according to claim 14, wherein the at least one elastomeric organopolysiloxane powder coated with silicone resin is present in an amount ranging from 3% to 7% by weight, relative to the total weight of the composition.

16. The composition according to claim 1, wherein the composition of combination (A) comprises a mixture of at least one elastomeric organopolysiloxane powder coated with silicone resin and of at least one uncoated elastomeric organopolysiloxane powder.

17. The composition according to claim 16, wherein the at least one uncoated elastomeric organopolysiloxane powder is present in an amount ranging from 1% to 10% by weight, relative to the total weight of the composition.

18. The composition according to claim 17, wherein the at least one uncoated elastomeric organopolysiloxane powder is present in an amount ranging from 3% to 7% by weight, relative to the total weight of the composition.

19. The composition according to claim 16, wherein the at least one elastomeric organopolysiloxane powder coated with silicone resin is present in an amount ranging from 1% to 10% by weight, relative to the total weight of the composition.

20. The composition according to claim 19, wherein the at least one elastomeric organopolysiloxane powder coated with silicone resin is present in an amount ranging from 3% to 7% by weight, relative to the total weight of the composition.

21. The composition according to claim 1, further comprising at least one spherical filler.

22. The composition according to claim 21, wherein the at least one spherical filler is chosen from organic fillers.

23. The composition according to claim 21, wherein the at least one spherical filler is chosen from polymethyl methacrylate powders, acrylic powders other than polymethyl methacrylate powders, and polyurethane powders.

24. The composition according to claim 21, wherein the at least one spherical filler is present in an amount ranging from 10% to 40% by weight, relative to the total weight of the composition.

25. The composition according to claim 24, wherein the at least one spherical filler is present in an amount ranging from 15% to 25% by weight, relative to the total weight of the composition.

26. The composition according to claim 1, wherein when the at least two powders are chosen from combination (A) or (C), the composition further comprises at least one polymethyl methacrylate powder.

27. The composition according to claim 1, wherein the at least one polymethyl methacrylate powder of (B) has a density ranging from 0.3 to 1.5.

28. The composition according to claim 27, wherein the at least one polymethyl methacrylate powder has a density ranging from 1 to 1.5.

29. The composition according to claim 1, wherein the at least one polymethyl methacrylate powder of (B) is present in an amount ranging from 5% to 20% by weight, relative to the total weight of the composition.

30. The composition according to claim 29, wherein the at least one polymethyl methacrylate powder is present in an amount ranging from 8% to 15% by weight, relative to the total weight of the composition.

31. The composition according to claim 1, wherein when the at least two powders are chosen from combination (A) or (B), the composition further comprises at least one acrylic polymer powder other than a polymethyl methacrylate powder.

32. The composition according to claim 1, wherein the at least one acrylic polymer powder other than a polymethyl methacrylate powder of (C) is chosen from powders of acrylonitrile polymer and powders of acrylonitrile copolymer.

33. The composition according to claim 32, wherein the at least one acrylic polymer powder comprises expanded hollow particles chosen from powders of acrylonitrile polymer and powders of acrylonitrile copolymer.

34. The composition according to claim 1, wherein the at least one acrylic polymer powder of (C) comprises particles with a mass per unit volume ranging from 15 kg/m³ to 200 kg/m³.

35. The composition according to claim 34, wherein the at least one acrylic polymer powder comprises particles with a mass per unit volume ranging from 60 kg/m³ to 80 kg/m³.

36. The composition according to claim 1, wherein the at least one acrylic polymer of (C) is chosen from copolymers comprising at least two of acrylonitrile, acrylic monomers, styrene monomers, and vinylidene chloride.

37. The composition according to claim 1, wherein the at least one acrylic polymer of (C) is a copolymer containing
from 0% to 60% of units derived from vinylidene chloride,
from 20% to 90% of units derived from acrylonitrile, and
from 0% to 50% of units derived from at least one of acrylic monomers and styrene monomers,
wherein the sum of the percentages by weight equals 100.

38. The composition according to claim 37, wherein the acrylic monomer is chosen from methyl (meth)acrylate and ethyl (meth)acrylate.

39. The composition according to claim 36, wherein the styrene monomer is chosen from α-methylstyrene and styrene.

40. The composition according to claim 1, wherein the at least one acrylic polymer powder other than a polymethyl methacrylate powder of (C) comprises at least one of hollow particles of an expanded copolymer of vinylidene chloride and of acrylonitrile and hollow particles of an expanded copolymer of vinylidene chloride, of acrylonitrile, and of methacrylate.

41. The composition according to claim 1, wherein the at least one acrylic polymer powder of (C) comprises particles having an internal cavity containing at least one gas chosen from air, nitrogen, isobutene, and isopentane.

42. The composition according to claim 1, wherein the at least one acrylic polymer powder of (C) comprises particles having a particle size ranging from 1 μm to 80 μm.

43. The composition according to claim 42, wherein the at least one acrylic polymer powder comprises particles having a particle size ranging from 10 μm to 30 μm.

44. The composition according to claim 1, wherein the at least one acrylic powder other than the polymethyl methacrylate powder of (C) is present in an amount ranging from 0.05% to 2% by weight, relative to the total weight of the composition.

45. The composition according to claim 44, wherein the at least one acrylic powder other than the polymethyl methacrylate powder of (C) is present in an amount ranging from 0.1% to 1.2% by weight, relative to the total weight of the composition.

46. The composition according to claim 1, wherein when the at least two powders are chosen from combination (A) or (C), the composition further comprises at least one polyurethane powder.

47. The composition according to claim 1, wherein the at least one polyurethane powder of (B) is a powder of a copolymer of hexamethylene diisocyanate and of trimethylol hexyl lactone.

48. The composition according to claim 1, wherein the at least one polyurethane powder of (B) is present in an amount ranging from 0.5% to 30% by weight, relative to the total weight of the composition.

49. The composition according to claim 48, wherein the at least one polyurethane powder is present in an amount ranging from 5% to 15% by weight, relative to the total weight of the composition.

50. The composition according to claim 1, wherein when the at least two powders are chosen from combination (B) or (C), the composition further comprises at least one N-acylamino acid powder.

51. The composition according to claim 1, wherein the at least one N-acylamino acid of (A) comprises an acyl group containing from 8 to 22 carbon atoms.

52. The composition according to claim 1, wherein the at least one N-acylamino acid of (A) comprises an acyl group chosen from 2-ethylhexanoyl, caproyl, lauroyl, myristoyl, palmitoyl, stearoyl, and cocoyl groups.

53. The composition according to claim 1, wherein the amino acid of the at least one N-acylamino acid of (A) is chosen from lysine, glutamic acid, and alanine.

54. The composition according to claim 1, wherein the at least one N-acylamino acid of (A) is lauroyllysine.

55. The composition according to claim 1, wherein the at least one N-acylamino acid powder of (A) is present in an amount ranging from 5% to 20% by weight, relative to the total weight of the composition.

56. The composition according to claim 55, wherein the at least one N-acylamino acid powder is present in an amount ranging from 8% to 15% by weight, relative to the total weight of the composition.

57. The composition according to claim 1, wherein the barium sulfate is coated with at least one N-acylamino acid.

58. The composition according to claim 57, wherein the barium sulfate is coated with lauroyllysine.

59. The composition according to claim 1, wherein the barium sulfate is present in an amount ranging from 1% to 10% by weight, relative to the total weight of the composition.

60. The composition according to claim 59, wherein the barium sulfate is present in an amount ranging from 3% to 7% by weight, relative to the total weight of the composition.

61. The composition according to claim 1, further comprising at least one pulverulent dyestuff.

62. The composition according to claim 61, wherein the at least one pulverulent dyestuff is chosen from pigments and nacres.

63. The composition according to claim 61, wherein the at least one pulverulent dyestuff is present in an amount ranging from 0.5% to 40% by weight, relative to the total weight of the composition.

64. The composition according to claim 63, wherein the at least one pulverulent dyestuff is present in an amount ranging from 3% to 25% by weight, relative to the total weight of the composition.

65. The composition according to claim 1, further comprising at least one additional filler.

66. The composition according to claim 65, wherein the at least one additional filler is chosen from talc, mica, silica, kaolin, polyamide powders, poly-β-alanine powders, polyethylene powders, tetrafluoroethylene polymer powders, starch, boron nitride, silicone resin powders, hydroxyapatite, sericite, glass beads, and ceramic beads.

67. The composition according to claim 65, wherein the at least one additional filler is present in an amount ranging from 0.5% to 75% by weight, relative to the total weight of the composition.

68. The composition according to claim 67, wherein the at least one additional filler is present in an amount ranging from 5% to 60% by weight, relative to the total weight of the composition.

69. The composition according to claim 1, wherein the composition is free of compounds chosen from calcium carbonate, magnesium carbonate, magnesium hydrocarbonate, chalks, titanium dioxide, and metal soaps derived from organic carboxylic acids containing from 8 to 22 carbon atoms.

70. The composition according to claim 1, wherein the composition comprises a total content of pulverulent compounds ranging from 80% to 99% by weight, relative to the total weight of the composition.

71. The composition according to claim 70, wherein the total content of pulverulent compounds ranges from 85% to 99% by weight, relative to the total weight of the composition.

72. The composition according to claim 1, further comprising at least one oil.

73. The composition according to claim 72, wherein the at least one oil is present in an amount ranging from 1% to 20% by weight, relative to the total weight of the composition.

74. The composition according to claim 73, wherein the at least one oil is present in an amount ranging from 2% to 15% by weight, relative to the total weight of the composition.

75. The composition according to claim 1, further comprising at least one cosmetic ingredient chosen from antioxidants, fragrances, preserving agents, neutralizers, surfactants, waxes, water, sunscreens, vitamins, moisturizers, self-tanning compounds, antiwrinkle active agents, and deodorant active agents.

76. The composition according to claim 1, wherein the composition is anhydrous.

77. The composition according to claim 1, wherein the composition is in a form chosen from loose powder and compact powder.

78. The composition according to claim 1, wherein the composition is chosen from makeup powder and skincare powder.

79. The composition according to claim 1, wherein the composition is in a form chosen from foundations, eyeshadows, makeup rouges, concealer products, body makeup products, face care products, body care products, and deodorant powders.

80. A process for making up and/or therapeutically treating the skin, comprising applying to the skin a cosmetic composition in powder form comprising
barium sulfate and
at least two powders chosen from one of the following combinations:
(A) at least one elastomeric organopolysiloxane powder and at least one N-acylamino acid powder;
(B) at least one polymethyl methacrylate powder and at least one polyurethane powder; and
(C) at least one acrylic polymer powder other than a polymethyl methacrylate powder.

81. A method of obtaining a deposit on natural skin which allows the grain of the skin to show through, comprising applying to the skin a cosmetic composition in powder form comprising
barium sulfate and
at least two powders chosen from one of the following combinations”
(A) at least one elastomeric organopolysiloxane powder and at least one N-acylamino acid powder;
(B) at least one polymethyl methacrylate powder and at least one polyurethane powder; and
(C) at least one acrylic polymer powder other than a polymethyl methacrylate powder.
